(19) United States
(12) Patent Application Publication
(10) Pub. No.: US 2026/0335620 A1
(43) Pub. Date: Oct. 29, 2026

(54) Medical and/or pharmaceutical fluid connector

(71) Applicant: RAUMEDIC AG, Münchberg (DE)

(72) Inventors: Eva-Maria HEISS, Regensburg (DE); Günter SCHOEBERLEIN, Helmbrechts (DE); Tobias FESTEL, Sparneck (DE)

(21) Appl. No.: 19/124,202

(22) PCT Filed: Oct. 26, 2023
(86) PCT No.: PCT/EP2023/079925

§ 371 Date: Apr. 24, 2025

(30) Foreign Application Priority Data
Oct. 26, 2022 (EP) 10 2022 211 360.8

(51) Int. Cl. A61M 39/10

(52) U.S. Cl. CPC A61M 39/105; A61M 2039/1088

(57) ABSTRACT

A medical and/or pharmaceutical fluid connector (1) serves to connect at least two fluid lines (2, 3, 4, 5). The fluid connector (1) has a main body (6) made of plastic by two-component injection moulding, with at least one plug-in main body portion (10) for plugged connection to at least one of the fluid lines (4, 5). The plug-in main body portion (10) has at least one outer circumferential seal (11, 12) for sealing the plug-in main body portion (10) against an inner wall of the fluid line (4, 5) that is to be connected. The plug-in main body portion (10) is designed as a hard component, and the seal (11, 12) is designed as a soft component, of the two-component injection-moulded main body (6). The plug-in main body portion (10) has at least two circumferential seals (11, 12) for sealing the plug-in main body portion (10) against coaxially extending inner walls of the fluid lines (4, 5) that are to be connected, and, radially between the circumferential seals (11, 12), at least one connection fluid channel (20, 20a) of the fluid connector (1; 25) opens out from the plug-in main body portion (10). This results in a medical and/or pharmaceutical fluid connector which, while being cost-effective to produce, is operationally safe and in particular is suitable for mass production.

The invention relates to a medical and/or pharmaceutical fluid connector for connecting at least two fluid lines.

A corresponding fluid connector is known on the market.

Applications for such a fluid connector include the hospital sector, the laboratory sector and the sector for pharmaceutical filling.

It is an object of the present invention to further develop a fluid connector of the above-mentioned type such that, while being cost-effective to produce, is operationally safe and in particular suitable for mass production.

According to the invention, this object is achieved by a medical and/or pharmaceutical fluid connector having the features specified in claim 1.

According to the invention, it was found that instead of separate sealing O-rings, for example, a soft component may be used as a sealing component, which is moulded onto a hard component of a two-component injection-moulded main body in a two-component injection moulding process.

This results in particular in a reduction in the number of parts of the connector, which on the one hand simplifies manufacture, in particular its logistics, and also makes the seal captive. The seal designed as a soft component is also especially operationally safe, as the only contact surface to be sealed is the abutment of the soft component on a wall of the medical fluid line to be sealed and connected. A sealed state between the seal and the main body portion of the main body is achieved anyway due to the two-component production. The plug-in main body portion on the one hand and the seal on the other may also be components of a multi-component injection-moulded main body. The main body, or even the entire fluid connector, may be accordingly manufactured as injection-moulded elements with more than two components.

In the context of this description, the term “fluid” can include both liquids and gases. The term can also include liquid-gas mixtures. For example, the fluid can comprise or be air. For example, air may flow in or through the medical fluid connector according to the invention.

The fluid connector may be used, for example, for eye operations, in particular for correcting a refractive cataract. The fluid connector may also be used for supply in connection to other kinds of surgery, for example.

A TPE as a soft component plastic material according to claim 2 has proven effective in practice. Alternatively, silicone or polyurethane (PUR) may also be used as the plastic material for the soft component.

ABS as a hard component plastic material according to claim 3 has proven effective in practice. Alternatively, polypropylene (PP), polyethylene (PE), polycarbonate (PC), polybutylene terephthalate (PBT) or polyether ether ketone (PEEK) may also be used for the hard component.

The fluid connector may comprise exactly one seal. At least two circumferential seals according to the invention, which are provided in further embodiments, enable the main body of the fluid connector to be sealed, in particular against coaxially extending inner walls of the fluid line to be connected. This allows, for example, two inlets and two outlets of medical fluid lines to be connected to one another. An axially lower end of the fluid connector, on which the circumferential seals are arranged, can, for example, be inserted into a socket of a medical and/or pharmaceutical apparatus. The axially lower end of the fluid connector may be plug-shaped. It may be designed as a plug, in particular as a coaxial plug. One of the seals may be arranged on a lower collar region of the axially lower end of the fluid connector and another of the seals may be arranged on an upper collar region of the axially lower end of the fluid connector, wherein the upper collar region has a larger diameter than the lower collar region.

In one design according to the invention, at least one connecting fluid channel of the fluid connector opens out from the plug-in main body portion radially between the circumferential seals. The plug-in main body portion of the two-component injection-moulded main body can thus have a compact design.

The fluid connector according to the invention can ensure a short-circuit-free connection to a fluid line, in particular a coaxial fluid line, since one of the circumferential seals serve to seal the inner line of the fluid line against an outer line of the fluid line that surrounds the inner line, and the other seal of the circumferential seals serve to seal the outer line of the fluid line to the outside.

In particular, the at least one connecting fluid channel may open out from the upper collar region and axially and radially above the lower collar region. The upper and lower collar regions may be substantially cylindrical.

A web between the two circumferential seals according to claim 7 facilitates an injection-moulded design of the two circumferential seals made of the plastic material of the soft component.

An overflow portion according to claim 10 facilitates the injection moulding of the seals from the soft component and ensures that sufficient moulding material is available for the circumferential seals of the soft component. Un-wanted trapped air can escape in a defined manner during the injection moulding process.

The at least two seals may be arranged on the fluid connector concentrically to one another, in particular in a cross-sectional plane through the fluid connector. The at least two seals may have different circumferences. In particular, the at least two seals have an annular shape, with the radius of the seals either increasing or decreasing along a longitudinal axis of the fluid connector or a connecting axis of the fluid connector with a fluid line. In particular, the radius of the seals may decrease or become smaller in an insertion direction of the plug-in main body portion of the fluid connector.

A two-component injection-moulded main body according to claim 15 allows fluid lines to be plugged onto the mating plug portions. This allows for a simple connection of the fluid connector to these fluid lines. The fluid connector may have exactly two such mating plug portions.

A design of the outer wall of the mating plug portion according to claim 16 without an undercut facilitates the removal from the mould in the injection moulding process.

An angle between the mating plug portions according to claim 17 makes it easier to plug fluid lines onto these mating plug plug-in portions, as the ends of the mating plug portions are spaced further apart and are therefore more easily accessible individually. If the two fluid lines to be plugged on have different functions, an intuitive individualization of the two mating plug portions can also be achieved in this way, avoiding undesirable confusion of the mating plug portions. The angle can also make the mating plug portions and the fluid lines in the fluid connector meet the overall installation space requirements. The angle at which the two mating plug portions extend in relation to one another may be greater than 15°. This angle is regularly less than 60°, less than 45° and may be less than 30°. The angle may be around 20°.

A two-component injection-moulded main body according to claim 18 has, as an alternative to the at least one mating plug portion, at least one mating socket portion, into which a fluid line can be inserted. Depending on the application, such a socket variant can offer advantages. An inner wall of the at least one mating socket portion can also be designed without an undercut, which offers corresponding advantages, as explained above in connection to claim 7. If several mating socket portions, for example two mating socket portions, are used, these can extend at an angle to one another, which corresponds to the one already explained above with reference to claim 8.

In particular, the fluid connector may be a medical fluid connector.

Example embodiments of the invention are explained in more detail below with reference to the drawings. In these:

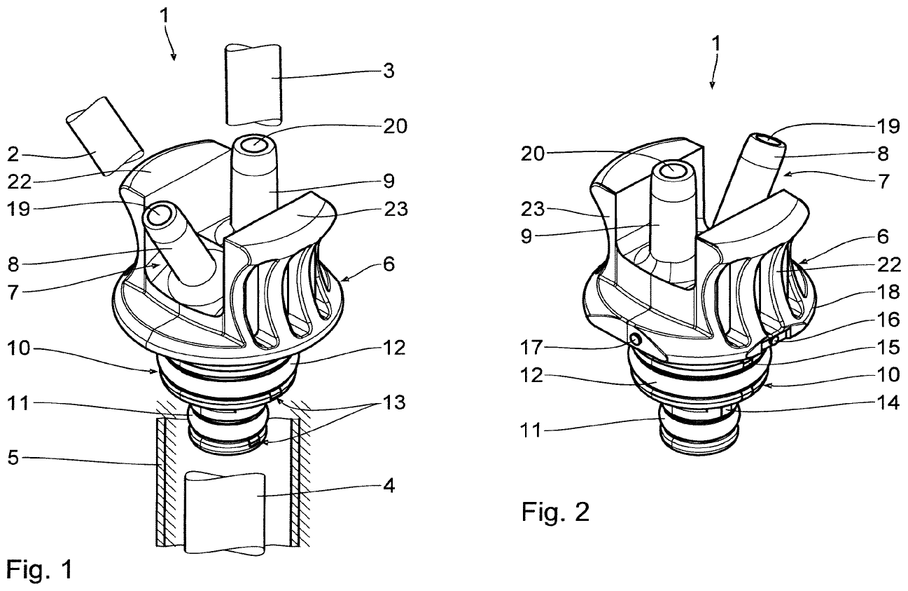

FIG. 1 shows a perspective view of an embodiment of a medical fluid connector for connecting at least two fluid lines, in the case of the illustrated embodiment for connecting a total of four medical fluid lines;

FIG. 2 shows the medical fluid connector according to FIG. 1, again in perspective view from the opposite direction compared to FIG. 1;

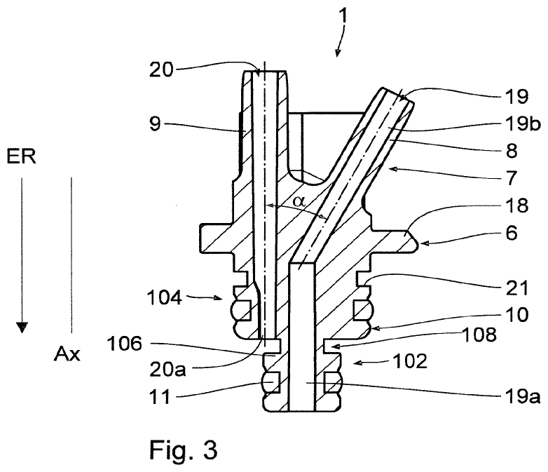

FIG. 3 shows a longitudinal sectional view through the medical fluid connector in a perspective view according to FIG. 2 to illustrate the extension of two connecting fluid channels between the four medical fluid lines to be connected;

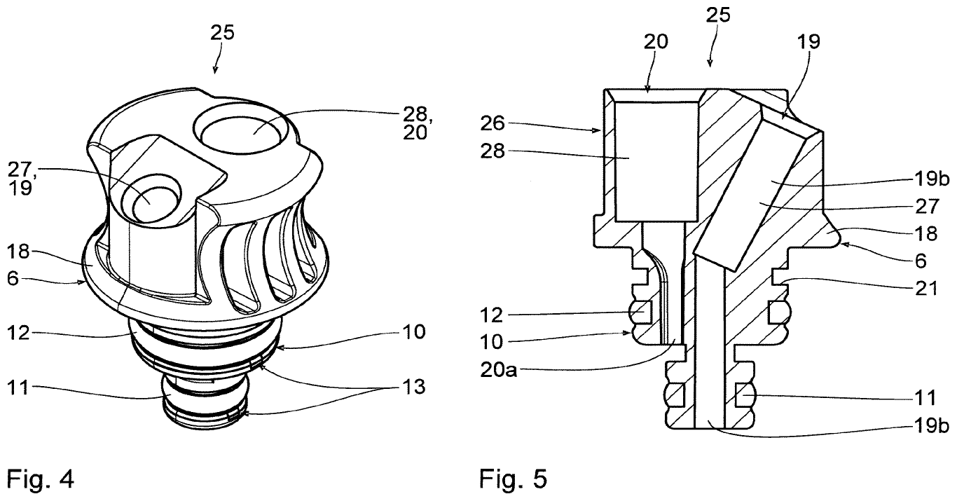

FIG. 4 shows another embodiment of a medical fluid connector for connecting at least two medical fluid lines, in the case of the illustrated embodiment for connecting a total of four medical fluid lines; and

FIG. 5 shows, in an illustration similar to FIG. 3, a longitudinal sectional view through the embodiment according to FIG. 4.

A medical fluid connector 1 (see FIG. 1) serves to connect at least two medical fluid lines, in the illustrated embodiment to connect four medical fluid lines. These four medical fluid lines to be connected to the fluid connector 1 are indicated in FIG. 1 as fluid lines 2, 3, 4 and 5, wherein the fluid lines 2, 3 are medical hose lines and the fluid lines 4 and 5 are a medical coaxial line with an inner lumen (fluid line 4) and a ring lumen (fluid line 5).

The fluid connector 1 has a two-component injection-moulded main body 6, which has a mating plug plug-in main body portion 7 for plugged connection to the fluid lines 2, 3 via mating plug portions 8, 9. The two-component injection-moulded main body 6 further comprises a plug-in main body portion 10 for plugged connection to the two other fluid lines 4, 5, i.e. with the medical coaxial line. The plug-in main body portion 10 has two external circumferential seals 11, 12. Seal 11 serves to seal the plug-in main body portion 10 against an inner wall of the fluid line 4 to be connected. Seal 12 serves to seal the plug-in main body portion against an inner wall of the fluid line 5 to be connected, i.e. against the outer inner wall of the ring lumen of the medical coaxial line 4, 5.

The plug-in main body portion 10 is designed as a hard plastic component and the seals 11, 12 are designed as soft plastic component of the two-component injection-moulded main body 6 of the medical fluid connector 1.

A thermoplastic elastomer (TPE) is used as the plastic material for the soft component. Other examples of soft components are silicone or polyurethane (PUR).

A thermoplastic polymer is also used as the plastic material for the hard component. One example of a material for the hard component is a thermoplastic terpolymer, in particular an acrylonitrile-butadiene-styrene copolymer (ABS). Further examples of the hard component are polypropylene (PP), polyethylene (PE), polycarbonate (PC), polybutylene terephthalate (PBT) or polyether ether ketone (PEEK).

Depending on the design of the medical fluid connector 1, it may, instead of the two seals 11, 12, also have exactly one seal, which is then designed as a soft component, for example if used to connect two medical fluid lines, i.e. one fluid line on the side of the mating plug plug-in main body portion 7 and one fluid line on the side of the plug-in main body portion 10.

The two seals 11, 12 each have an overflow portion 13 (see FIG. 1), in which excess soft component injection moulding material may accumulate during the injection moulding process of the two-component injection moulding main body 6.

The two circumferential seals 11, 12 are formed and arranged concentrically in relation to one another. As shown in FIG. 1, the seals 11, 12 are arranged one above the other along the longitudinal axis of the fluid connector. The radius of the upper seal 12 is larger than the radius of the lower seal 11. The seal 11 is, in the longitudinal direction of the fluid connector, located on a lower collar region 102 of the plug connector main body portion 10, which is designed to be arranged in the inner fluid line 4 when connected to a coaxial fluid line 4, 5, in particular to be pushed into the inner fluid line 4. The seal 12 is, in the longitudinal direction of the fluid connector, located on an upper collar region 104 of the plug connector main body portion 10, which is arranged above the lower collar region 102 in the longitudinal direction of the fluid connector and which is designed to be arranged in the outer fluid line 5 when connected to a coaxial fluid line 4, 5, in particular to be pushed into the outer fluid line 5 (see FIG. 3). The lower and upper collar regions 102, 104 form the lower end of the fluid connector and the plug connector body portion 10, respectively.

The inlet or outlet of the fluid line 4 is for this purpose offset in relation to the inlet or outlet of the fluid line 5.

The two circumferential seals 11, 12 are connected to one another via a web 14 (see FIG. 2) made of the soft component. The seal 12 is connected to a soft component injection port 16 of the two-component injection-moulded main body 6 via another web 15.

At the same axial height as the soft component injection port 16, a hard component injection port 17 of the two-component injection-moulded main body 6 is arranged such as to be offset in the circumferential direction.

At the height of the two injection ports 16, 17, the two-component injection-moulded main body 6 has a circumferential flange 18 made of the hard component. The flange 18 may serve as a stop for the medical coaxial line 4, 5.

The flange 18 is formed as a portion or part projecting from the two-component injection-moulded main body. In a longitudinal section through the fluid connector (see, for example, FIG. 3), the flange 18 comprises two projections protruding or projecting from the two-component injection-moulded main body 6.

The two mating plug portions 8, 9 each have an outer wall without undercuts. The mating plug portions 8, 9 may be fixed to the plugged-on fluid lines 2, 3 by means of solvent adhesion, i.e. in an adhesive manner.

The two mating plug portions 8, 9 extend at an angle of 20° to one another. This angle is designated with a in FIG. 3. Depending on the design of the fluid connector 1, this angle between the longitudinal axes of the mating plug portions 8, 9 may also have a different absolute value of at least 10° or more than 10°. This angle is regularly less than 60°.

FIG. 3 illustrates an extension of connecting fluid channels 19, 20 in the two-component injection-moulded main body 6. In FIG. 3, the longitudinal axis of the fluid connector is shown in particular by the solid line Ax and the insertion direction of the fluid connector or the plug-in main body portion 10, respectively, by the arrow ER.

The connecting fluid channel 19 connects the fluid lines 2 and 4 to one another. In the plug-in main body portion 10, the connecting fluid channel 19 initially extends in a first channel portion 19a coaxially to a longitudinal axis of the two-component injection-moulded main body portion 10. Axially, approximately at the height of the flange 18, the connecting fluid channel 19 then bends and extends at an angle of 20° with respect to the longitudinal axis along a channel portion 19b and opens out from the main body 6 via the mating plug portion 8. An internal diameter of the connecting fluid channel 19 is in the range between 1.5 mm and 4 mm, for example in the range between 2 mm and 2.5 mm.

The connecting fluid channel 20 connects the two fluid lines 3 and 5. The connecting fluid channel 20 extends parallel to the longitudinal axis of the main body 6. The connecting fluid channel 20 opens out from the plug-in main body portion 10 radially between the two circumferential seals 11, 12 via an opening out portion 20a. In the area of this opening out portion 20a, an internal diameter of the connecting fluid channel 20 may be reduced compared to the other internal diameter, which is as large as the internal diameter of the connecting fluid channel 19.

As shown in FIG. 3, the lower collar region 102, which forms the axially lower end of the main body 6 or the plug-in main body portion 10, respectively, has a circumferential recess or notch, in which the seal 11 is arranged and fixed. The upper collar region 104, which is arranged axially above the lower collar region 102, also has a circumferential recess or notch, in which the seal 12 is arranged and fixed. In an axial view, the upper collar region 104 is located between the lower collar region 102 and the flange, or stop 18. In the longitudinal direction of the fluid connector, the plug-in main body portion 10 comprises a circumferential undercut or recess/notch or groove 108 between the lower collar region 102 and the upper collar region 104. The circumferential groove 108 is shaped such that a step 106, by which the lower collar region 102 is offset from the upper collar region 104, is formed in the lower collar region 102. The opening out portion 20a is arranged axially and radially above the step 106.

In other words, if the fluid connector and a coaxial fluid line are in a connected state, the connecting fluid channel 20 and the outer line 5 of the fluid line as well as the connecting fluid channel 19 and the inner line 4 of the fluid line are connected to one another in a fluid-tight manner.

The fluid connector shown in FIG. 3 also comprises at least one undercut 21. The undercut 21 may extend around the main body 6. The undercut 21 may be formed as an indentation or recess in the main body 6. The at least one undercut 21 is arranged between the flange 18 and the seal closest to the flange 18, i.e. seal 12. The at least one undercut 21 is formed and/or arranged on the main body 6 such that a latching element, which may be arranged on the coaxial fluid line 4, 5, can latch or engage with the undercut 21 and thus secure the connection between the coaxial fluid line and the fluid connector. The latching element may be a spring element, for example.

FIGS. 4 and 5 show another embodiment of a medical fluid connector 25, which may be used instead of the medical fluid connector 1 according to FIGS. 1 to 3. Components and functions explained above in connection to the embodiment according to FIGS. 1 to 3 have the same reference numerals and are not discussed again in detail.

Instead of a mating plug plug-in main body portion with mating plug portions, the fluid connector 25 has a mating socket plug-in main body portion 26 with mating socket portions 27, 28. The mating socket portion 27 serves as a socket for plugged connection to the fluid line 2, i.e. it has a function corresponding to that of the mating plug portion 8. The mating socket portion 28 serves as a socket for plugged connection to the fluid line 3, i.e. it has a function corresponding to that of the mating plug portion 9. In the fluid connector 25, the fluid lines 2, 3 are therefore not plugged onto corresponding plug portions, but are inserted into corresponding socket portions, i.e. the mating socket portions 27, 28.

Inner walls of the mating socket portions 27, 28 are designed without undercuts and are used for solvent adhesion to fix the fluid lines 2, 3 in accordance with the above explanation regarding the mating plug portions 8, 9.

An angle between the two mating socket portions 27, 28 corresponds to the angle discussed above in connection with the mating plug portions 8, 9. In the illustrated example embodiment, the angle between the mating socket portions 27, 28 is again 20°.

In the area of the mating socket portions 27, 28, an inner diameter of the connecting fluid channels 19, 20 for receiving the fluid lines 2, 3 is increased compared to the remaining inner diameter. This remaining internal diameter may in turn range from 1 mm to 5 mm, as already explained above in connection with the connecting fluid channels 19, 20 of the fluid connector 1.

The seals 11, 12 ensure a process-reliable seal between the plug-in main body portion 10 and the corresponding inner walls of the coaxial line 4, 5 designed as a coaxial socket. A circumferential undercut 21 interacts here with a latching element of this coaxial line 4, 5 to axially secure the coaxial line 4, 5 to the plug-in main body portion 10.

The two-component injection-moulded main body 6 also comprises two actuating wings 22, 23, which may be used to grip the main body 6 when it is inserted into the pulled out of the coaxial line 4, 5. The actuating wings 22, 23 may, for example, be designed as ridges extending substantially in the longitudinal direction of the main body 6. The actuating wings 22, 23, in particular the ridges, may be arranged in the circumferential direction of the main body 6. The ridges may have an outer geometry that is bent inwards or curved in relation to the main body 6. The actuating wings 22, 23 may extend between the flange 18 and the mating plug portions 7, 8 or mating socket portions 27, 28.

This description further relates to the following aspects:
1. A medical fluid connector (1; 25) for connecting at least two medical fluid lines (2, 3, 4, 5),
   comprising a two-component injection-moulded main body (6) made of plastic with at least one plug-in main body portion (10) for plugged connection to at least one of the fluid lines (4, 5), wherein the plug-in main body portion (10) has at least one outer circumferential seal (11, 12) for sealing the plug-in main body portion (10) against an inner wall of the fluid line (4, 5) that is to be connected,
   wherein the plug-in main body portion (10) is designed as a hard component, and the seal (11, 12) as a soft component, of the two-component injection-moulded main body (6).
2. The medical fluid connector according to aspect 1, characterized in that a TPE is used as the plastic material for the soft component.
3. The medical fluid connector according to aspect 1 or 2, characterized in that ABS is used as the plastic material for the hard component.
4. The medical fluid connector according to one of the aspects 1 to 3, characterized in that the plug-in main body portion (10) has at least two circumferential seals (11, 12) for sealing the plug-in main body portion (10) against coaxially extending inner walls of the fluid lines (4, 5) that are to be connected.
5. The medical fluid connector according to aspect 4, characterized in that, radially between the circumferential seals (11, 12), at least one connection fluid channel (20, 20a) of the fluid connector (1; 25) opens out from the plug-in main body portion (10).
6. The medical fluid connector according to aspect 4 or 5, characterized in that the two circumferential seals (11, 12) are connected to one another via a web (14) made of the soft component.
7. The medical fluid connector according to one of the aspects 1 to 6, characterized in that the at least one outer circumferential seal (11, 12) has an overflow portion (13), in which excess injection moulding material can accumulate.
8. The medical fluid connector according to one of the aspects 1 to 7, characterized in that the two-component injection-moulded main body (6) comprising a mating plug plug-in main body portion (7) with at least one mating plug portion (8, 9) for plugged connection to at least another one (2, 3) of the fluid lines (2 to 5).
9. The medical fluid connector according to aspect 8, characterized in that the at least one mating plug portion (8, 9) comprises an outer wall without undercuts.
10. The medical fluid connector according to aspect 8 or 9, characterized by two mating plug portions (8, 9) for plugged connection to one of the other fluid lines, wherein the two mating plug portions (8, 9) extend at an angle (α) of more than 10° with respect to one another.
11. The medical fluid connector according to one of the aspects 1 to 7, characterized in that the two-component injection-moulded main body (6) comprises a mating-socket plug-in main body portion (26) with at least one mating socket portion (27, 28) for plugged connection to at least another one (2, 3) of the fluid lines (2 to 5).

1-19. (canceled)

20. A medical and/or pharmaceutical fluid connector for connecting at least two medical fluid lines,
comprising a main body with at least one plug-in main body portion for plugged connection to at least one of the fluid lines, wherein the plug-in main body portion has at least one outer circumferential seal for sealing the plug-in main body portion against an inner wall of the fluid line that is to be connected,
wherein the plug-in main body portion has at least two circumferential seals for sealing the plug-in main body portion against coaxially extending inner walls of the fluid lines that are to be connected and, radially between the circumferential seals, at least one connection fluid channel of the fluid connector opens out from the plug-in main body portion,
characterized in that
the main body is a two-component injection-molded main body made of plastic;
the plug-in main body portion is designed as a hard component, and the seal as a soft component molded onto the hard component, of the two-component injection-molded main body, and
the seals are captive.

21. The fluid connector according to claim 20, wherein a TPE is used as the plastic material for the soft component.

22. The fluid connector according to claim 20, wherein ABS or PP is used as the plastic material for the hard component.

23. The fluid connector according to claim 20, wherein the two circumferential seals are connected to one another via a web made of the soft component.

24. The fluid connector according to claim 20, wherein the circumferential seals are arranged at an axially lower end of the fluid connector.

25. The fluid connector according to claim 24, wherein one of the seals is arranged on a lower collar region of the axially lower end of the fluid connector, and another of the seals is arranged on an upper collar region of the axially lower end of the fluid connector, the upper collar region having a larger diameter than the lower collar region.

26. The fluid connector according to claim 24, wherein the axially lower end of the fluid connector is designed as a plug, in particular as a coaxial plug.

27. The fluid connector according to claim 25, wherein the at least one connecting fluid channel opens out from the upper collar region and axially and radially above the lower collar region.

28. The fluid connector according to claim 25, wherein the lower and upper collar regions are substantially cylindrical.

29. The fluid connector according to claim 20, wherein the at least two outer circumferential seals have an overflow portion, in which excess injection molding material can accumulate.

30. The fluid connector according to claim 20, wherein the at least two seals are arranged on the fluid connector concentrically to one another, in particular in a cross-sectional plane through the fluid connector.

31. The fluid connector according to claim 20, wherein the at least two seals have different circumferences.

32. The fluid connector according to claim 20, wherein the at least two seals are annular shaped, wherein the radius of the seals either increases or decreases along a longitudinal axis of the fluid connector or a connection axis of the fluid connector with a fluid line.

33. The fluid connector according to claim 32, wherein the radius of the seals decreases or becomes smaller in an insertion direction (ER) of the plug-in main body portion of the fluid connector.

34. The fluid connector according to claim 20, wherein the two-component injection-molded main body comprises a mating plug plug-in main body portion with at least one mating plug portion for plugged connection to at least another one of the fluid lines.

35. The fluid connector according to claim 34, wherein the at least one mating plug portion comprises an outer wall without undercuts.

36. The fluid connector according to claim 34, wherein the fluid connector further comprises two mating plug portions for plugged connection to one of the other fluid lines, wherein the two mating plug portions extend at an angle (α) of more than 10° with respect to one another.

37. The fluid connector according to claim 20, wherein the two-component injection-molded main body comprises a mating socket plug-in main body portion with at least one mating socket portion for plugged connection to at least another one of the fluid lines.

38. The fluid connector according to claim 20, wherein the fluid connector is a medical fluid connector.
